(19) United States
(12) Patent Application Publication
(10) Pub. No.: US 2008/0221645 A1
(43) Pub. Date: Sep. 11, 2008

(54) Neurotrophic Electrode Neural Interface Employing Quantum Dots

(75) Inventors: Philip R. Kennedy, Duluth, GA (US); Steven A. Siebert, Atlanta, GA (US)

Correspondence Address: BRYAN W. BOCKHOP, ESQ.;BOCKHOP & ASSOCIATES, LLC, 2375 MOSSY BRANCH DR., SNELLVILLE, GA, 30078, US

(73) Assignee: NEURAL SIGNALS, INC., Duluth, GA (US)

(21) Appl. No.: 12/042,742

(22) Filed: Mar. 5, 2008

Related U.S. Application Data
(60) Provisional application No. 60/893,161, filed on Mar. 6, 2007.

(51) Int. Cl. A61N 1/05

(52) U.S. Cl. 607/60; 977/950

(57) ABSTRACT

A device for interfacing neurons includes a substrate that defines at least one via passing therethrough. The via is configured to allow at least one neurite to grow therethrough. A light generating unit is disposed adjacent to the substrate and is configured to generate light of a predetermined frequency when an action potential from the neurite is sensed. A light sensor that is spaced apart from the substrate is configured to assert a neural signal corresponding to the action potential when the light generating unit generates light of the predetermined frequency.

### BACKGROUND OF THE INVENTION

1. Field of the Invention

The present invention generally relates to a neural interface and, more specifically, to a transcutaneous neural interface.

2. Description of the Prior Art

Muscle paralysis affects over one hundred thousand people in the United States and approximately one million people worldwide. One class of patients who face severe difficulties in their daily lives is those with locked-in syndrome. Locked-in syndrome patients generally have a cognitively intact brain and a nearly completely paralyzed body. They are alert but cannot move or talk. They face a life-long challenge to communicate. Some patients may use eye movements, blinks or remnants of muscle movements to indicate binary signals, such as “yes” or “no.” One approach used to provide assistance to patients with locked-in syndrome has been described in U.S. Pat. No. 4,852,573, which is hereby incorporated by reference (see, e.g., col. 5, 11. 39 et seq. for a discussion of use of nerve growth factor to enhance nerve growth into an electrode). In this approach, an electrode is implanted into the patient's brain and signals from the electrode may be used to control an electronic device.

Neural interfaces may be implanted in human subjects for communication and motor restoration applications. An ideal invasive neural interface would include multiple recording sites, isolation of each recording site to one axon to avoid cross-talk, mechanical stability, biocompatibility, long term recording capabilities, and a size minimization.

The ability to record neural activity over long durations is critically dependent upon proper neural interface design. Current approaches involve silicon microelectronic machining techniques for controllability and size reduction of passive electrodes. The desire for multiple recording sites has led to the development of multi-electrode arrays with corresponding high-density micro-ribbons. However these devices have not been able to achieve long term recording due to signal degradation and artifacts due to gliosis and micro-movements of the components.

One type of electrode recording system that has been available for long term human implantation is the neurotrophic electrode, which includes hollow glass cone containing gold recording wires that allow recording from axons grown into the glass cone under the influence of neural trophic factors. Recording from this reconstituted neuropil has produced action potentials (APs) that display robust signal-to-noise ratios over long time periods. The recording system uses transcutaneous FM transmission of the amplified system, thereby avoiding the need for wires. It is powered by air gap induction coils, obviating the need for batteries. This system has been implanted in six locked-in humans to provide them with control of a switch or a computer cursor, thus restoring synthetic speech, Internet access, environmental control, and other applications.

However, existing applications have implanted electrodes in only one or a few sites. This limited number of implanted sites limits the amount of information that can be transmitted to the external interface.

An increase in the number of electrode sites and size reduction of the recording area for individual axon isolation could substantially increase the likelihood of success in such applications as speech synthesis. The current neurotrophic electrode has a cone diameter of 20-25 μm and can contain anywhere between 10-50 individual neurites. Reducing the diameter into the 1-5 μm range would effectively limit the number of neurites grown into the device. The increase of neural data obtained from multiple recording sites would require additional wiring from the device to the amplifier system located outside of the skull. By applying a wireless method of transmitting signals from the neural interface to the electronics mounted on the skull, a high throughput of data can be achieved without the introduction of bulky micro-ribbons.

Direct optical imaging of neural activity has been demonstrated through voltage sensitive or Ca 2+-sensitive dyes with a charge coupled device (CCD) camera for detection. The CCD camera system allows simultaneously recording of multiple neural activities over a surface area at up to 5 kHz resolution. However, as the entire cortical surface area is bathed with the dye, there is no selectivity over the neurons being observed. The CCD camera is used to monitor electrical activity of all neurons over a 2-dimension visual field. This approach is not appropriate for long term recordings because of bleaching and phototoxic effects of the dyes.

Quantum dots have received substantial attention for biological marking applications utilizing photoluminescence, where higher energy light induces a characteristic (lower energy) quantum dot light emission. The electric dipole created at the quantum dot during optical light adsorption may be large enough to stimulate or inhibit neuronal firing. Quantum dots offer an advantage of increased efficiency. This is attributed to the inverse square relationship between charge and distance. By using either antibody-antigen recognition or peptide recognition groups, the quantum dots may be bound to the neuron and separated by less than 10 nanometers, as opposed to micron ranges in current neural interfaces.

The long term stability of such an approach has yet to be seen. Quantum dots (QDs) are mostly formed from Cd based heterostructures and although they are used routinely in vivo for marking applications, there are still existing questions as to their toxicity. Organic capping layers have been created to envelop the quantum dots, however these layers tend to act as electrical traps that increase efficiency for optical labeling applications, but reduce efficiency of opto-electrical conversion. In addition, quantum dots do not form long term interfaces, typically only lasting a matter of weeks. Tethered quantum dot films have been studied to address the question of stability but these films degraded within 3-5 days in primary neuron cultures. While cell binding techniques allow precise cell selection, they do not provide a method that can control interface construction at specified site areas.

Therefore, there is a need for a stable neural interface that transmits local neural action potentials from a plurality of recording sites.

### SUMMARY OF THE INVENTION

The disadvantages of the prior art are overcome by the present invention which, in one aspect, is a device for interfacing neurons that includes a substrate that defines at least one via passing therethrough. The via is configured to allow at least one neurite to grow therethrough. A light generating unit is disposed adjacent to the substrate and is configured to generate light of a predetermined frequency when an action potential from the neurite is sensed. A light sensor that is spaced apart from the substrate is configured to assert a neural signal corresponding to the action potential when the light generating unit generates light of the predetermined frequency.

In another aspect, the invention is a neural interface for interfacing to a cerebral cortex of a patient in a region subtended by a portion of the patient's skull. The neural interface includes a neurotrophic electrode array. The neurotrophic electrode array includes a substrate, an amplifier and a plurality of quantum dots. The substrate defines a plurality of spaced-apart vias. Each via is treated with a neurotrophic factor that stimulates neurites to grow into the via when the neurotrophic electrode array is juxtaposed adjacent to the cerebral cortex. Each amplifier is adjacent to each via receives power from a thermoelectric generator and generates an amplified signal that corresponds to an action potential generated by a neurite that has grown into a corresponding via. The plurality of quantum dots are responsive to the amplified signal and emit a photonic signal in response to the amplified signal. A sensing and transmitting unit is spaced apart from the neurotrophic electrode array includes a charge-coupled device and a transmitter. The charge-coupled device senses light from each of the plurality of quantum dots and generates an electrical signal corresponding to the photonic signal. The transmitter is responsive to the electrical signal and generates a radio-frequency signal indicative of a state of the electrical signal.

In yet another aspect, the invention is a method of communicating an action potential to a receiving device, in which light of a predetermined frequency is emitted in response to an assertion of the action potential from a neurite. The light is sensed. Upon sensing the light, a wireless signal corresponding to the action potential is transmitted to the receiving device.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram of a quantum dot neural electrode sensing system.

FIG. 2 is a schematic diagram of a quantum dot neural electrode array sensing system.

FIG. 3 is a schematic diagram of a frequency sensitive quantum dot neural electrode array sensing system.

FIG. 4 is a top plan view of a quantum dot neural electrode array.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

Quantum dots possess a number of intrinsic and useful properties that are useful if integrated into neural interface devices. The emission wavelengths of quantum dots can be controlled by the size of the particles. Manufacturing of colloidal quantum dots is relatively simple and cost-effective compared to other optoelectronic devices. Embedding the quantum dots in polymers optimizes charge transport for electroluminescent devices.

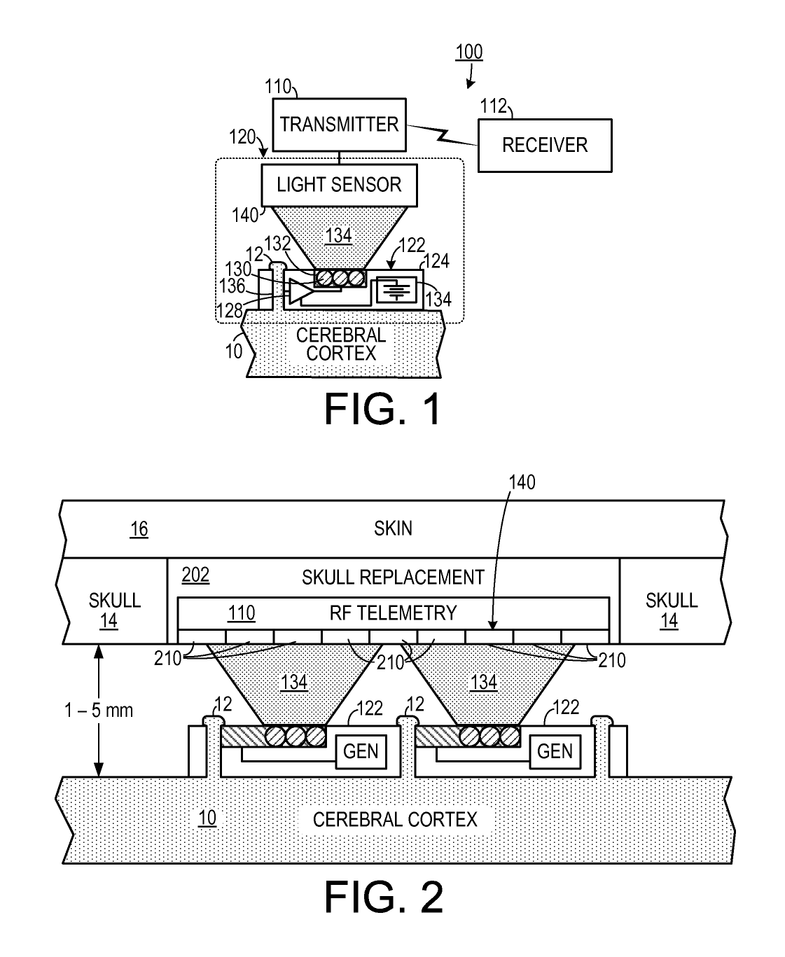

As shown in FIG. 1, one exemplary embodiment of a neural interface 100 includes an action potential sensing system 120 that senses neural action potentials and generates a signal representative thereof. A transmitter 110 transmits the signal wirelessly to a remote receiver 112, which may then transmit the signal to a processor for use in such applications as device control and speech synthesis.

The action potential sensing system 120 includes a light generating unit 122 (or electrode) that is placed against a predetermined location of the patient's cortex 10 and that senses an action potential from a neurite 12 that extends from the cortex 10. The light generating unit 122 includes a substrate 124 that defines at least one via 136 (or hole) passing therethrough. An amplifier 128 senses action potentials asserted by the neurite 12 and generates an amplified signal corresponding to the action potentials. At least one quantum dot 130 (which may be enveloped in a polymer 132 to stabilize the quantum dot 130) generates light 134 of a predetermined frequency in response to the signal from the amplifier 128. The quantum dots 130 are excited by the electrical signal from the amplifier 128 and could include a fluorescent crystal of materials such as cadmium. A light sensor 140 that is spaced apart from the quantum dots 130 senses the light 134 and transmits a signal representative thereof to the transmitter 110. The neurite 12, having grown through the via 136 maintains the stability of the light generating unit 122 with respect to the cerebral cortex 10.

The amplifier 128 is powered by a power source 134, which in one embodiment includes a thermoelectric generator. The thermoelectric generator (such as a thermocouple) is a device that generates electricity in response to a temperature differential between the cerebral cortex 10 and the skin of the patient's head.

A portion of a simple quantum dot neural electrode array is shown in FIG. 2. Each neurite 12 has a different light generating unit 122 associated therewith. The light sensor 140 may include an array of charge coupled devices (CCD) 210 and may be placed with the transmitter 110 in a skull replacement component 202, which is placed in a hole in the patient's skull 14. The transmitter 110 may be powered in a manner similar to that of radio-frequency ID (RFID) tags, in which electromagnetic energy is transmitted to the device, which is then converted to electrical power. Once placed, the skin 16 may be sutured over the skull replacement component 202.

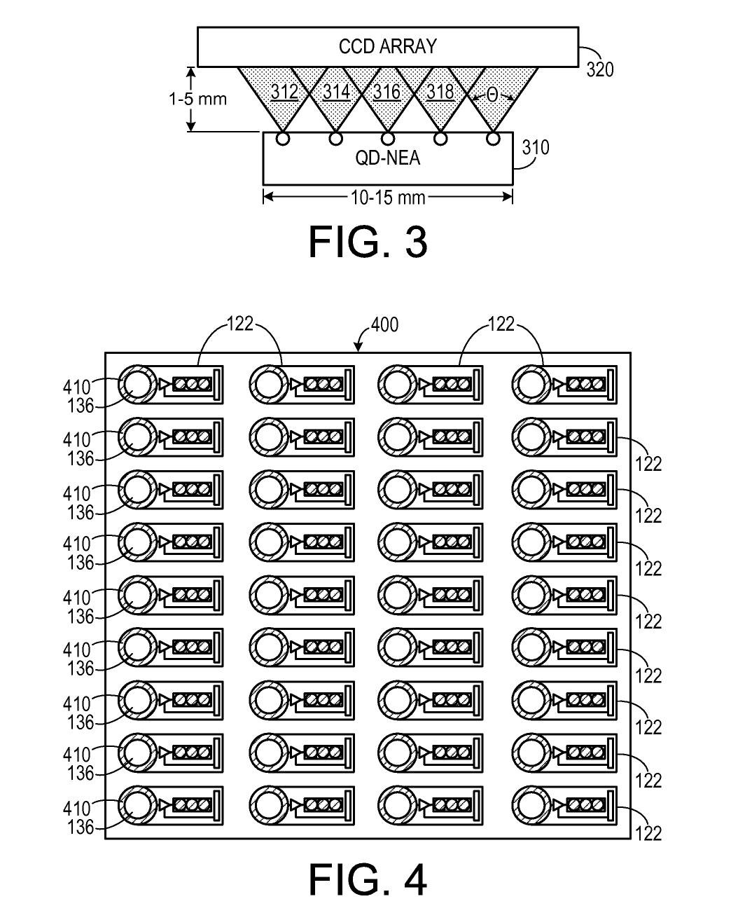

In one embodiment, as shown in FIG. 3, a quantum dot neural electrode array 310 may include groups quantum dots that each generate light of a different frequency (312, 314, 316, 318, etc.) Each quantum dot group is placed so as to be exited by a different neurite. The CCD array 320 can sense the different frequencies. This allows precise differentiation of the action potentials generated by the different neurites. The light frequency of can be selected by selecting the size of the quantum dots being placed next to a via.

A top view of a quantum dot neural electrode array 400 is shown in FIG. 4. The array 400 includes a plurality of sensor members 122. In one embodiment, each via 136 may be treated with a neurotrophic factor 410 to promote the growth of neurites into the via 136.

The emission angle, ⊖, is determined in terms of efficiency. A quantum dot will emit light isotropically, that is in all directions. Only a small percentage of the emitted photons will be effective for CCD detection. Assuming the device will operate as a typical Lambertian emitter, a cosine function determines light intensity versus emission angle. The function is N=Nₒ cos ⊖, where N is the radiant intensity, Nₒ is the radiance normal to the emitting surface, and ⊖ is the emission angle. While the amount of detected light is effectively limited by the emission angle, this amount can be doubled by creating a reflective surface under the quantum dot light-emitting layer. If the signal intensity is limited to between 90-100% received at the CCD for useful data, ⊖ is then 25.9°. For separation distances between 1-3 mm, the range of areas of incident light onto the CCD array 320 from a quantum dot is determined to be between 0.209 mm² and 1.88 mm² respectively using trigonometric calculations.

The same calculations may be used to determine separation between the quantum dots. By spacing out the quantum dots on the array, local CCD sections are assigned to each QD. This makes signal processing simpler. Limiting the emission efficiency to 25% corresponds to quantum dot separation of 1.5-4.65 mm. A quantum dot neural electrode array with 10-15 mm diameter corresponds to areas of 78.5-177 mm². These ranges will allow an average of 31 quantum dot sets in the array for a 1-1.5 cm² device. This separation of the quantum dots also allows room for the MOSFET amplification and thermoelectric circuitry.

An advantage of the CCD approach is its ability for error correction. The implanted device will have vertical micro-movements associated with brain pulsations and general micro-movements transverse to the array. Whereas this would lead to a loss of signal in standard IR photodetectors and even low level RF signaling, in a large enough CCD array standard 2 dimensional signal processing will be able to track the movement of the quantum dot neural electrode array constantly. The CCD array can be constructed with this in mind; its area would be the area of the underlying QD-NEA plus additional area on the perimeter for the expected movement. A 4 cm² CCD array area could be sufficient. Vertical movement can be accounted for by assigning a few quantum dots to emit constantly a specific NIR frequency for continual monitoring and calibration.

Charge couple devices (CCD) are well known for low light detection applications, such as astronomical studies. The range of light detection ranges from UV to infrared and they are capable of single photon detection. The efficiency of photons converted to electrons can be determined from the dark current of the device. This is equivalent to electrical noise created in an environment without light. The light flux received and converted to an electrical current should be more than the dark current to be measured. Commercial CCD systems use 60 Hz for integration, but higher frequencies may be needed in monitoring neural activity. This is because the detection sampling of the CCD arrays should be made less than the time scale of the action potential. The Nyquist frequency, the lowest sampling frequency still able to accurately sample the waveform, would be 2 kHz to monitor action potential components up to 1 kHz, a time scale of 1 ms.

CCD arrays converted for neural recording operate at frequencies between 1-5 kHz which limits the effect of dark current; an integration time of 300 (3333 Hz) had a dark current noise of only 5 electrons. For a 2 kHz system this would correspond to 8 electrons. If the current is to be larger than the dark noise by a factor of 10, the minimum CCD current is then 2.56×10⁻¹⁴ A (using I=dq/dt).

For a typical CCD element with quantum efficiency of 50%, 160 photons should be received at the detector every 0.5 ms for 2 kHz. It is recognized a large portion of the emitted light, approximately 97%, will be directed in directions away from the detector for an emission angle of 25.9°. This results in a transmission efficiency of 3%. The electroluminescence quantum efficiency is about 0.52%. This results in total system efficiency of 0.0078%, which results in a required current of 3.28⁻¹⁰ A to be generated at the quantum dots for successful transmission of neural signals. A thermoelectric generator supplying 1-5 V 1-10 μW, is able to supply a current from 200 nA to 1 μA. Considering the supply to operate MOSFET circuitry and quantum dots in parallel, the current then is equally split between each quantum dot for a total of 600 possible devices. This is more than ample power to operate the averaged array of 31 devices determined in the above calculations.

In one experimental embodiment the quantum dot—neurotrophic electrode array (QD-NEA) was approximately 20-50 μm thick and 1 cm in diameter. It was constructed from thinned, flexible silicon with polymer components. The inherent flexibility, durability, and minimum weight in this design were important, as the device is meant to be placed on the cortex surface. The vias were about 1-5 μm in diameter and the action potentials were measured and amplified with built in MOSFET circuitry.

The amplified signals were used to drive quantum dots to emit infrared light at specified frequencies assigned to each neuron. The intensity of the emitted light was directly related to the action potential voltage. The signals were then detected and sorted with a CCD array built into a skull replacement component.

The quantum dot neural electrode array structure disclosed herein is able to address these difficulties of specifying areas for interfacing and long term stability and toxicity control. In the quantum dot neural electrode array, the neurites are encouraged to grow into each electrode via, rather than having quantum dots applied directly to a cell body. The quantum dots are encapsulated in polymer packaging which is able to both protect them from the biological environment and confine any toxicity from escaping into the environment.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A device for interfacing neurons, comprising:
a. a substrate defining at least one via passing therethrough, the via configured to allow at least one neurite to grow therethrough;
b. a light generating unit, disposed adjacent to the substrate, that is configured to generate light of a predetermined frequency when an action potential from the neurite is sensed; and
c. a light sensor, spaced apart from the substrate, that is configured to assert a neural signal corresponding to the action potential when the light generating unit generates light of the predetermined frequency.

2. The device of claim 1, further comprising a neurotrophic factor applied to the via.

3. The device of claim 1, further comprising a transmitter coupled to the light sensor that is configured to transmit a remote signal corresponding to the neural signal to a remote receiver.

4. The device of claim 3, wherein the transmitter comprises a wireless transmitter.

5. The device of claim 1, wherein the light generating unit comprises:
a. a power source;
b. an amplifier that receives power from the power source and that is configured to amplify the action potential from the neurite so as to generate an electrical signal representative thereof, and
c. at least one quantum dot, responsive to the electrical signal from the amplifier, that is configured to emit light of the preselected frequency when the electrical signal is asserted.

6. The device of claim 5, wherein the power source comprises a thermoelectric generator.

7. The device of claim 5, wherein the quantum dot has a size selected so as to cause the quantum dot to emit light of the preselected frequency.

8. The device of claim 5, wherein the quantum dot comprises a fluorescent crystal.

9. The device of claim 5, further comprising a polymer envelope that surrounds the quantum dot.

10. The device of claim 1, wherein the light sensor comprises a charge coupled device.

11. The device of claim 1, wherein the substrate comprises a plurality of vias, and further comprising a plurality of light generating units, wherein each light generating unit is disposed adjacent to a different one of the plurality of vias.

12. The device of claim 11, wherein each light generating unit is configured to emit light of a different predetermined frequency.

13. The device of claim 12, wherein the light sensor is configured to sense light of each different predetermined frequency.

14. The device of claim 12, wherein the light sensor is configured to assert a different value of the neural signal in response to sensing light of each different predetermined frequency.

15. The device of claim 12, wherein the light sensor comprises an array of charge coupled devices.

16. A neural interface for interfacing to a cerebral cortex of a patient in a region subtended by a portion of the patient's skull, comprising:
a. a neurotrophic electrode array that includes:
i. a substrate defining a plurality of spaced-apart vias, each via treated with a neurotrophic factor that stimulates neurites to grow into the via when the neurotrophic electrode array is juxtaposed adjacent to the cerebral cortex;
ii. a thermoelectric generator;
iii. an amplifier adjacent to each via that receives power from the thermoelectric generator and that generates an amplified signal that corresponds to an action potential generated by a neurite that has grown into a corresponding via; and
iv. a plurality of quantum dots that are responsive to the amplified signal and that emit a photonic signal in response to the amplified signal; and
b. a sensing and transmitting unit, spaced apart from the neurotrophic electrode array, that includes:
i. a charge-coupled device that senses light from each of the plurality of quantum dots and that generates an electrical signal corresponding to the photonic signal; and
ii. a transmitter that is responsive to the electrical signal and that generates a radio-frequency signal indicative of a state of the electrical signal.

17. The neural interface of claim 16, wherein the plurality of quantum dots are each assigned to a quantum dot group, wherein each quantum dot group is disposed adjacent to a different via and wherein each of the quantum dot groups is configured to emit light of a different frequency.

18. The neural interface of claim 17, wherein each of the quantum dot groups is enclosed in a polymer envelope.

19. A method of communicating an action potential to a receiving device, comprising the actions of:
a. emitting light of a predetermined frequency in response to an assertion of the action potential from a neurite;
b. sensing the light; and
c. upon sensing the light, transmitting a wireless signal corresponding to the action potential to the receiving device.

20. The method of claim 19, wherein the action of emitting light comprises the actions of:
a. amplifying the action potential so as to generate an electrical signal; and
b. exciting a quantum dot with the electrical signal.
